(19) United States
(12) Patent Application Publication
(10) Pub. No.: US 2019/0287670 A1
(43) Pub. Date: Sep. 19, 2019

(54) A METHOD AND SYSTEM FOR PROVIDING TREATMENT PROTOCOLS FOR PATIENTS

(71) Applicant: Aslam KHAN, Mississauga (CA)

(72) Inventors: Aslam KHAN, MISSISSAUGA (CA); Mayar ABBASI, PIERREFONDS (CA)

(21) Appl. No.: 16/301,974

(22) PCT Filed: May 10, 2017
(86) PCT No.: PCT/CA2017/000123

§ 371 Date: Nov. 15, 2018

Related U.S. Application Data
(60) Provisional application No. 62/336,692, filed on May 15, 2016.

(51) Int. Cl. G16H 20/30; G06Q 30/02; G16H 30/40

(52) U.S. Cl. CPC G16H 20/30; G16H 30/40; G06Q 30/0283

(57) ABSTRACT

A method and system for monetizing a treatment protocol is provided, as is a method for standardizing a treatment protocol. The method for monetizing the protocol comprises a remote server receiving a request for a patient record from a health care provider, allowing the health care provider access to the patient record, receiving a request for a treatment protocol from the health care provider, the remote server providing the treatment protocol to a treatment device, the treatment device conducting the treatment protocol, and the remote server assessing and recording a fee to the health care provider.

### CROSS REFERENCE TO RELATED APPLICATIONS

This application claims benefit of United States Provisional Patent Application Ser. No. 62,336,692, filed on May 15, 2016 and entitled A METHOD AND SYSTEM FOR PROVIDING TREATMENT PROTOCOLS FOR PATIENTS, the contents of which are incorporated herein by reference.

### FIELD

The present technology relates to a processor controlled method to transform X-ray images into a treatment protocol. More specifically, the technology relates to determining, providing, storing, and monetizing the providing and storing of treatment protocols for patients suffering from spine related pain.

### BACKGROUND

The implementation of computerized equipment, computerized medical information systems and network or internet communications has allowed for great advances in treating patients. For one, a patient need not be treated in the same location by the same health care provider, if, in another location, a second health care provider can access the medical records. U.S. Pat. No. 5,911,687 discloses a wide area medical information system and a method using thereof comprising a wide area network, a plurality of doctor terminals and patient terminals connected to the wide area network, and a management server including at least an electronic case record file storing clinic information for patient's and a doctor database storing data of a plurality of doctors, wherein the system searches the doctor database on the basis of patient information including the condition of the disease of a certain patient input from the patient terminal, selects the corresponding doctor, requests that the selected doctor take charge of examination and treatment for the aforementioned certain patient, registers the correspondence between the approved doctor and the aforementioned certain patient in the electronic case record file, gives the right to access the clinic information of the patient to the approved doctor, and executes the online examination and treatment via the doctor terminal and patient terminal, so that a patient existing in a wide area can receive remote examination and treatment services of high satisfaction and medical treatment related services other than examination and treatment without depending on the location. This system relies upon the doctor to determine what steps in treatment need to be taken and therefore it is not assured that the patient will receive the same treatment, and indeed, the same diagnosis in different locations.

Similarly, U.S. Pat. No. 5,915,240 discloses a computer system and method for accessing medical information over a network. The system partitions the functioning of the system between a client and server program optimized in a manner to assure synchronization of the master medical information databases on the servers with the local medical information database on the client, minimize the use of network resources, and allow new types of medical information to be easily included in the system. A server site on the network maintains a description of its medical information, as well as the most current and up-to-date medical reference information. The client program maintains a local database that is automatically synchronized over the network with revisions and new medical information, and provides a user with an interface to fully review the information in the database. The system also uses a context-sensitive call facility so that users of the Medical Lookup Reference program can easily get further expert assistance about the medical topic. The call feature uses the network connection to establish a conversation between the user and a person at a help site specified by the type of medical information they are currently referencing. Once a connection is established, the system allows the user to engage in a conversation with the person at the help site, and a record of the conversation can be saved in a database for auditing purposes. This system simply allows access to medical information. It does not ensure consistent diagnosis or consistent treatment.

An alternative approach is disclosed in U.S. Pat. No. 6,055,506. An outpatient care data system dedicated to the transmission, storage and retrieval of outpatient data relating to care of outpatients is provided with a regional data system located at a regional location, a plurality of metropolitan area data systems operatively connected to the regional data system, each of the metropolitan area data systems being located at a different metropolitan location. Each metropolitan area data system may be provided with an electronic nursing station located within a hospital and first and second types of outpatient systems operatively coupled to the electronic nursing station on a real-time basis. The first type of outpatient system is situated at a first non-hospital location remote from the hospital and includes a medical device associated with an outpatient present at the first non-hospital location, and the second type of outpatient system is situated at a second non-hospital location remote from the hospital and includes a medical device associated with an outpatient present at the second non-hospital location. While the system can store X-rays and other medical images and can include a medical device for treating or collecting patient data, it does not provide a controlled method to transform X-ray images into a treatment protocol and ensure that the patient receives a controlled and reproducible treatment in any given clinic.

User access and privacy issues are important considerations when patient records are shared. Further, if there is a desire to monetize the access, means are necessary to either track access, restrict access or both. U.S. Pat. No. 6,038,563 discloses a system and method for restricting database access to managed object information using a permissions table that specifies access rights corresponding to user access rights to the managed objects. An access control database has access control objects that collectively store information that specifies access rights by users to specified sets of the managed objects. The specified access rights include access rights to obtain management information from the network. An access control server provides users access to the managed objects in accordance with the access rights specified by the access control database. An information transfer mechanism sends management information from the network to a database management system (DBMS) for storage in a set of database tables. Each database table stores management information for a corresponding class of managed objects. An access control procedure limits access to the management information stored in the database tables using at least one permissions table. A permissions table defines a subset of rows in the database tables that are accessible to at least one of the users. The set of database table rows that are accessible corresponds to the managed object access rights specified by the access control database. A user access request to access management information in the database is intercepted, and the access control procedure is invoked when the user access request is a select statement. The database access engine accesses information in the set of database tables using the permissions tables such that each user is allowed access only to management information in the set of database tables that the user would be allowed by the access control database to access. This system only controls access and does not provide a treatment protocol to a treatment device and associate access with monetization of treatment.

In U.S. Pat. No. 6,041,411, a different method is used, in which user access rights are defined and verified. A method is provided for minimizing the potential for unauthorized use of digital information, particularly software programs, digital content and other computer information, by verifying user access rights to electronically transmitted digital information. A second computer system transmits requested digital information to a requesting first computing system in wrapped form, which includes digital instructions that must be successfully executed, or unwrapped, before access to the digital information is allowed. Successful unwrapping requires that certain conditions must be verified in accordance with the digital instructions, thereby allowing access to the digital information. In one embodiment, verification includes locking the digital information to the requesting computer system by comparing a generated digital fingerprint associated with the digital information to a digital fingerprint previously generated which is unique to the requesting computer system. This system only controls access and does not provide a treatment protocol to a treatment device and associate access with monetization of treatment.

Monetization, in its simplest form, relates to patient billing. Monetization of computer-based interactions is a relatively new business. Despite this, there are literally thousands of published patent application directed to monetization. One example is United States Patent Application 20110264532, which discloses a social advertising platform. Methods and systems for facilitating a social advertising platform are provided. According to one embodiment, a method is provided for enabling sharing of content by members of a peer-to-peer social advertising service that also promotes companies, products, services, organizations, charities and/or causes which they endorse. Profile information is maintained for each member and includes a list of brands, products, services, companies, organizations, charities or causes recommended or endorsed by the member. A shared link is generated corresponding to a destination link through which a member can share content associated with the destination link with third parties via social media. Responsive to receiving a click-through event for the shared link, the requestor is redirected to a social advertisement containing advertising content selected based upon the list of things endorsed by the member. After temporarily displaying the advertising content, the requestor is then redirected to the destination link. This system only tracks access and monetizes access to a social advertising platform. It does not provide a treatment protocol to a treatment device and associate access with monetization of treatment.

Another example is United States Patent Application 20110112911, which discloses a method and system for enabling consumer-creators to create advertisements on behalf of advertisers on an electronic network includes the uploading or creating of an ad on a given website by a consumer-creator and offering the ad to advertisers or publishers or visitors for presentation on various medium (electronic or otherwise) where the ad can be available on the given website or linked to the given website from another website. The method can enable the selection of an ad (that has been uploaded or created by the consumer-creator) by an advertiser or a publisher or visitor. The method can also broker a relationship among the consumer-creator, visitor, advertiser, or publisher. The method can also track usage of the ad and automatically enable payment for the use of the ad based on the relationship brokered and the use tracked of the ad. This system only tracks access and monetizes creation of advertising. It does not provide a treatment protocol to a treatment device and associate access with monetization of treatment.

In U.S. Pat. No. 7,805,377, an information record infrastructure, system and method is disclosed that includes billing. A method for controlling access to a medical record of a patient hosted by at least one medical record repository, comprising a plurality of sub-records, each sub-record having an associated different patient-controlled access control criteria, comprising: receiving, by an intermediary, a request for a medical record from a requester, said request comprising a medical record identifier, a requester identifier, requester authentication information, and patient-provided access control authorization; automatically processing, by the intermediary, the request for the medical record to authenticate the requester and determine sufficiency of the patient-provided access control authorization to meet the patient-controlled access control criteria for each respective sub-record encompassed by the request; and selectively communicating, from the intermediary to the at least one medical record repository, an identification of each sub-record for which access control criteria are determined to be sufficient for access by the requestor. An electronic payment authorization associated with the request may be generated, for compensation of at least one of the intermediary and a medical record repository. This system only tracks access to medical records and monetizes the access. It does not provide a treatment protocol to a treatment device and associate access with monetization of treatment.

In some instances, it may be preferable to allow access to patient records, but disallow access to treatment protocols. This requires a partitioning of information. If treatment is to be monetized, treatments necessarily should trigger billing, or assessment of a service fee or the like. Further partitioning is required if multiple health care providers are able to access the records as privacy rules would dictate that one health care provider does not see the billings or royalties paid by another health care provider. They should, however, be able to see the number of treatments, notes on the patient and the like, in order to optimize patient care. There should also be controls or standardization to ensure that treatment at one location will be the same as treatment at another location. It is an objective of the present technology to provide such interactions.

### SUMMARY

The present technology provides a method and system to allow the developer of treatment devices and treatment protocols to provide them to locations world-wide and to ensure that the quality of treatment and consistency of treatment is maintained from location to location. As development of devices and software to determine treatment protocols is costly, and the locations are widespread, it is useful to develop failsafe methods for monetizing the technologies. Hence, a method for monetizing a treatment protocol is provided that inherently provides consistency in treatment. The method comprises: a remote server receiving a request for a patient record from a health care provider, the remote server comprising a database; allowing the health care provider access to the patient record; receiving a request for a treatment protocol from the health care provider; the remote server providing the treatment protocol to a treatment device; the treatment device conducting the treatment protocol; and the remote server assessing the health care provider a fee for the treatment and recording the fee; wherein the database is partitioned such that the patient record and the treatment protocol are retained in separate parts of the database and each part has different access control criteria thereby restricting the health care provider's access to the treatment protocol.

Receiving the request comprises receiving a patient identifier, a health care provider identifier, a location identifier, and authentication information; allowing access comprises processing the identifiers and authentication information and providing access based on the access control criteria; and providing the treatment protocol comprises identifying the treatment device based on the location identifier and selectively communicating the treatment protocol to the treatment device.

In the specific instance wherein there is more than one health care provider in more than one location, the database is partitioned such that the health care providers share access to the patient data, cannot access the treatment protocol, and can only access their own assessed fees, based on different access control criteria. The fee may be a service fee.

The health care professional may also be involved in inputting patient data into a client and the client sending the data to an analytics server for processing into the treatment protocol prior to a request being made, wherein inputting patient data comprises identifying on a user interface, a plurality of points on frontal, side and top x ray images of the patient's skull.

In a specific situation, the method may further comprise determining a mean axis of rotation.

The preferred treatment device is a spinal and upper cervical impulse device.

A system configured to monetize a treatment protocol requested by a health care professional is also provided. The system comprises: a communication link; a treatment device; a remote server for receiving a request from the health care professional for a patient record, providing the patient record to the health care professional and providing the treatment protocol to the treatment device via the communication link, the remote server comprising a database and a processor, wherein the database is partitioned such that the patient record and the treatment protocol are retained in separate parts of the database and each part has different access control criteria thereby restricting the health care professional's access to the treatment protocol; a client, the client comprising a user interface and a communication module for communication with the remote server via the communication link, to allow the health care professional to send the request to access the patient record, to send a request to the remote server for the treatment protocol and to receive a bill associated with the client for the treatment.

The client is preferably configured to provide a patient identifier, a health care professional identifier, a location identifier, and authentication information and the processor in the remote server is configured to process the identifiers and authentication information, provide access based on the access control criteria, identify the treatment device based on the location identifier and selectively communicate the treatment protocol to the treatment device.

In the specific instance wherein there is more than one health care professional in more than one location, the database is partitioned such that more than one health care professional shares access to the patient data, cannot access the treatment protocol, and can only access their own fees assessed, based on different access control criteria.

In one monetization model, the processor is configured to assess a service fee.

The system may further comprise an analytics server for processing the patient data into the treatment protocol.

If required, the user interface comprises a display and a mouse for identifying a plurality of points on frontal, side and top x ray images of a patient's skull.

In specific situations, the processor is configured to determine a mean axis of rotation.

The treatment device is preferably a spinal and upper cervical impulse device.

The communication link may be a wireless communication link, which in turn, may be the internet. The opportunity exists for the data to be stored in a cloud.

In another embodiment a method of monetizing a treatment protocol is provided, the method comprising: identifying a patient with patient identifiers; collecting data from x-rays of a patient by a user clicking on a series of landmarks in the x-rays; sending the patient identifiers and data to an analytics server for processing; processing the data to determine a confidential treatment protocol; associating the confidential treatment protocol with the patient identifiers and the user; storing the confidential treatment protocol in a remote database; providing the confidential treatment protocol to a treatment device in response to the user's request; recording the treatment at the remote database and assessing a fee to the user in response to the treatment, wherein the database is partitioned to allow the user access to only the patient identifiers, patient data and a record of the number of treatments.

In one situation, the series of landmarks are a plurality of points on frontal, side and top x ray images of the patient's skull.

In a specific situation, the method comprises determining a mean axis of rotation.

The treatment device used in the method is preferably a spinal and upper cervical impulse device.

In another embodiment, a method of providing a standard, personalized treatment protocol to a number of treatment locations is provided. The method comprises generating patient data and patient records including patient identifiers in one location, sending the patient records and patient data to a remote server and an analytics server, storing patient records and patient data on the remote server, the analytics server processing patient data into the treatment protocol that is standardized for a patient, allowing access to the patient data and receiving requests for the treatment protocol from the treatment locations, providing the treatment protocol to a treatment device in a given treatment location, and recording the treatment on the remote server.

More specifically, receiving the request comprises receiving a patient identifier, a health professional identifier, a location identifier, and authentication information; allowing access comprises processing the identifiers and authentication information and providing access based on the access control criteria; and providing the treatment protocol comprises identifying the treatment device based on the location identifier and selectively communicating the treatment protocol to the treatment device.

The method preferably comprises monetizing the treatment protocol. Specifically, monetizing comprises the remote server assessing and recording a fee for the treatment location.

### FIGURES

FIG. 1 is a block diagram of the system for patient management and treatment of the present technology.

FIG. 2 is a block diagram of the method of the present technology for a new patient.

FIG. 3 is a block diagram of the method of the present technology for a returning patient.

FIG. 4 is a block diagram of a method of the present technology for determining a treatment protocol.

FIG. 5 is a block diagram of a continuation of the method of the present technology for determining a treatment protocol.

FIG. 6 is a block diagram of the architecture of the system.

### DESCRIPTION

Except as otherwise expressly provided, the following rules of interpretation apply to this specification (written description, claims and drawings): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, The terms “comprising,” “having,” “including,” and “containing” are to be construed as open ended terms (i.e., meaning “including, but not limited to,”) unless otherwise noted.

To the extent necessary to provide descriptive support, the subject matter and/or text of the appended claims is incorporated herein by reference in their entirety.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Cloud refers to cloud computing which is the use of computing resources (hardware and software) that are delivered as a service over a network (typically the Internet).

MRI refers to Magnetic Resonance Imaging.

CT refers to Computed Tomography scanning.

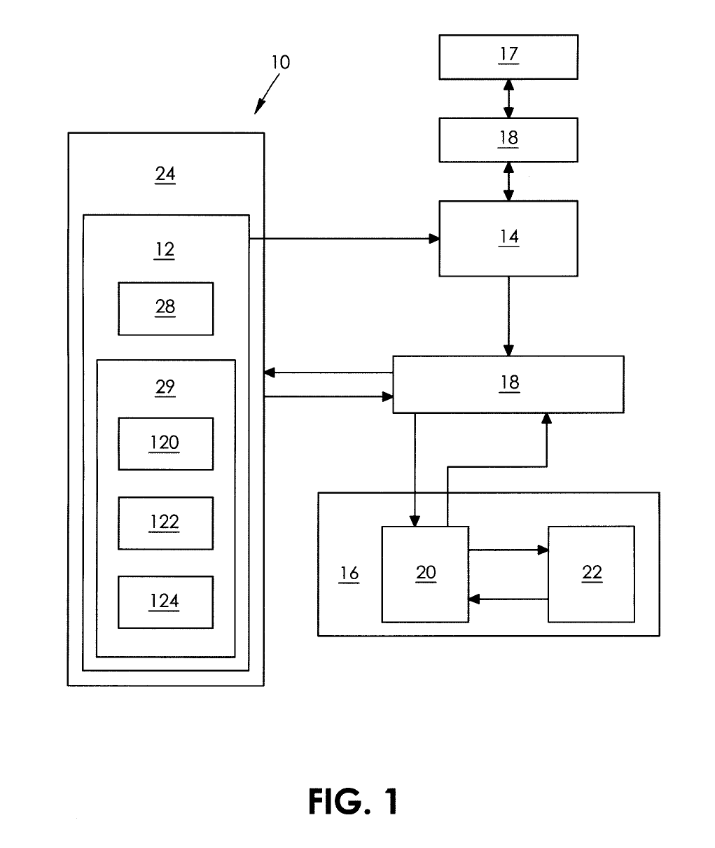

A system for patient management and treatment, generally referred to as 10 is shown in FIG. 1. The system has four main components, a client 12, an application 14, a database server 16 and an analytics server 17. The client 12 and database server 16 are in communication through the application 14 and via a secure wireless or wired communication link 18. The database server 16 is a remote server. The database server 16 has a patient management application 20 that manages the following: patient details (name, address, etc); patient identification number; patient visit details; patient x-rays; patient analysis results; patient treatment details; patient notes; and service fee assessed. Hence, the database server 16 stores all the patient data and billing in a database 22. This database 22 is in communication via the patient management application 20 with all treatment centres 24 by means of the secure wireless or wired communication link 18. The analytics server 17 is in communication through the application 14 via the secure wireless or wired communication link 18. The treatment centre 24 has a user interface 28 and a treatment module 29. As shown in FIG. 1, treatment data for the patient is directed specifically to the treatment module 29 from the analytics server 17. The treatment module has firmware 120, a user interface 122 and a treatment device 124.

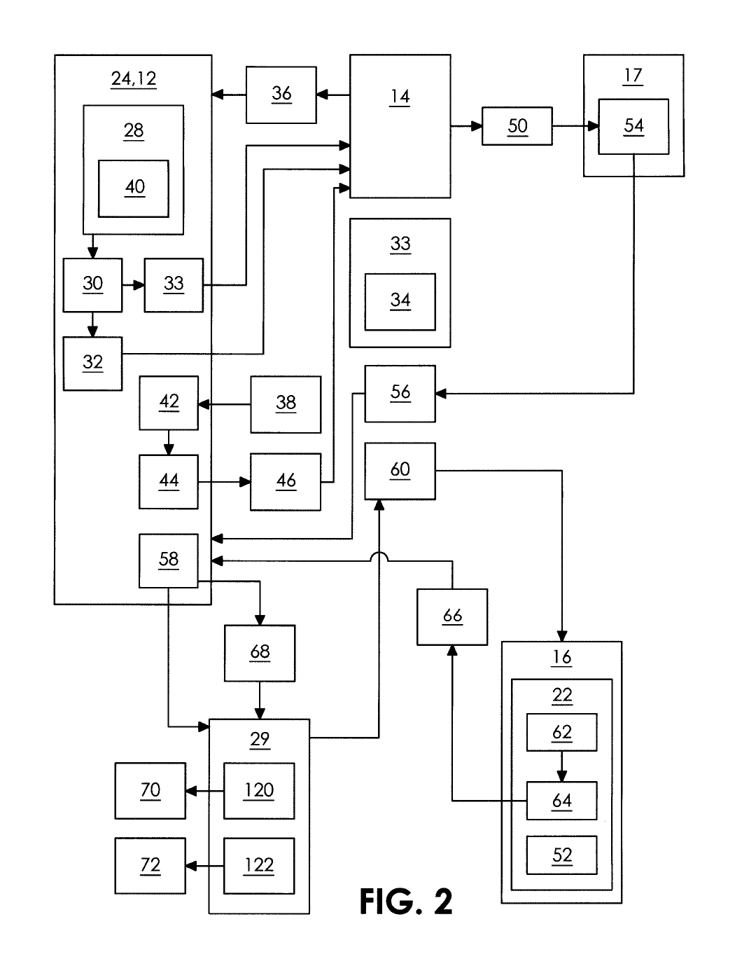

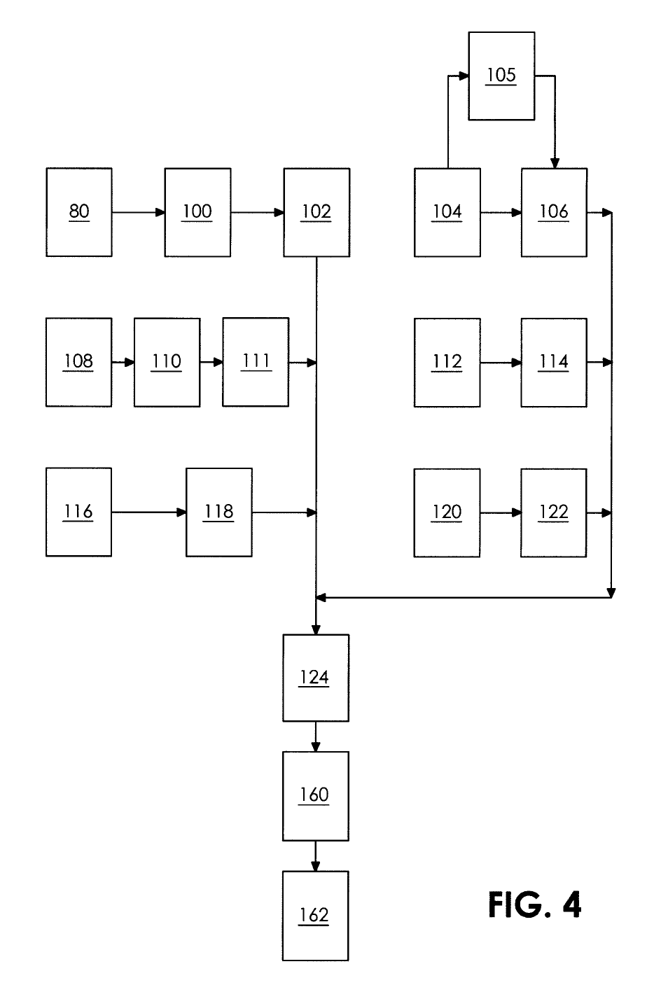

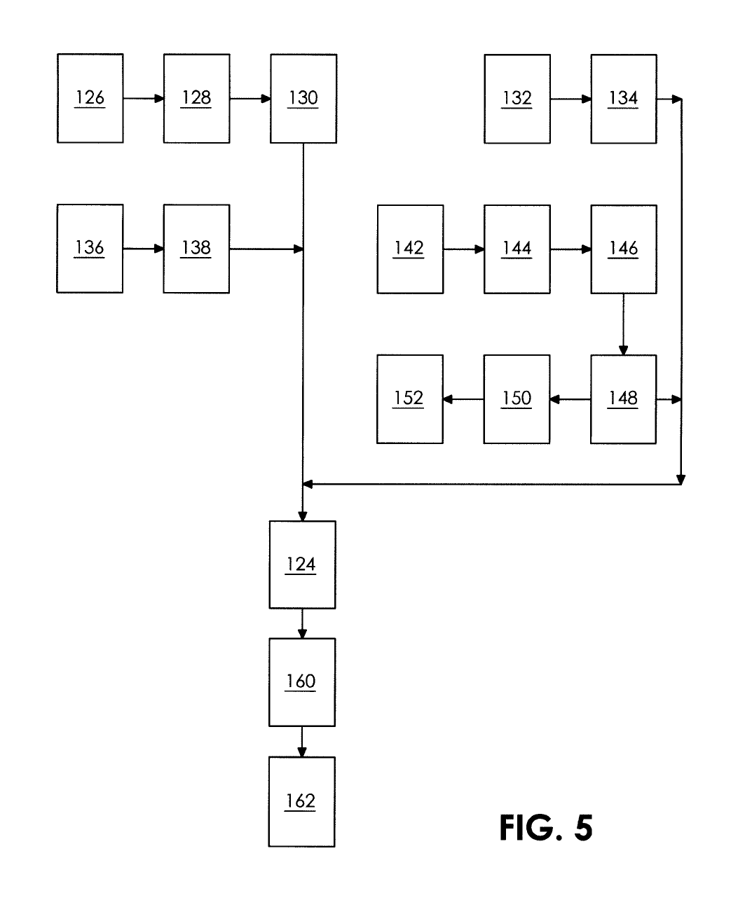

As shown in FIG. 2, an attending professional (health care provider) is required to log into 30 the system 10 at a treatment centre 24. If the patient is a new patient, new patient data are entered 32 and if the patient is a returning patient, the data are updated 33 as needed. The x-rays, or other images are entered 34 automatically from the imaging machine 33 (for example, but not limited to x-ray, MRI, CT). The application calls 36 the client 12 and sends 38 the x-rays. The user interface 28 will display 40 the x-rays or scans. The professional identifies by clicking 42 appropriate points of interest on the x-rays images, and the client software creates 44 data that are sent 46 to the application 14, as detailed in FIGS. 4 and 5. The data and x-rays are then sent 50 to the analytics server 17. The x-rays are stored 52 in the database 22 and the data are processed 54 by the analytics server 17. The processed data are then sent 56 back to the client 12 for use.

The client 12 then interface 58 with the treatment module 29. When the client 12 interfaces with the treatment module 29, a use is registered 60 for both the treatment centre and the patient. The use is stored 62 in the database 22. This triggers 64 a service fee that is subsequently charged 66 to the treatment centre 24. The client 12 instructs 68 the firmware 120 in the treatment module 29 to provide 70 a treatment. The treatment module user interface 122 displays 72 positioning information to the attending professional so that they can assist in positioning the treatment head, as needed. The treatment device then provides the treatment. The attending professional does not have access to the processed data, nor the specific treatment to be delivered, thereby ensuring that all treatments are recorded and the service fee charged.

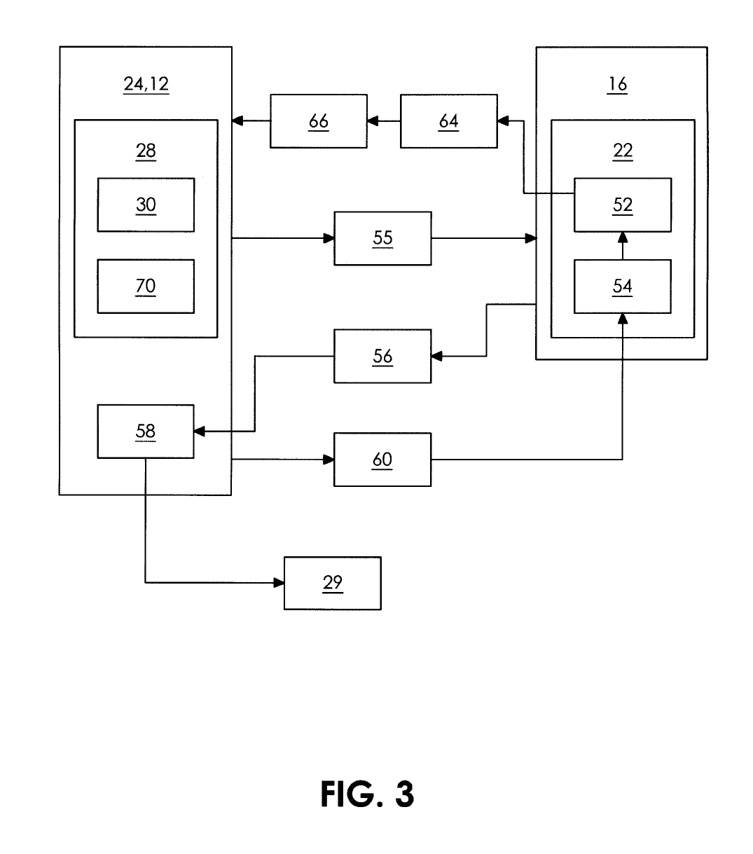

Henceforth, as shown in FIG. 3, when the patient returns for treatment, the attending professional logs in 30 at the treatment centre 24. The patient identification number is entered 70 using the user interface 28, the client 12 instructs 55 the database server 16 to send data, and the processed data are then sent 56 back to the client 12 for use. The client 12 then interfaces 58 with the treatment module 29. When the client 12 interfaces with the treatment module 29, a use is registered 60 for both the patient and for the treatment centre and is stored 62 in the database 22 at the database server 16. This triggers 64 a service fee that is subsequently charged 66 to the treatment centre 24. The attending professional does not have access to the processed data, nor the specific treatment to be delivered. This partitioning of data ensures that all treatments are recorded and the service fee charged. Sharing between locations is possible, with regard to the patient data, but not the service fee charged, or fees levied.

The treatment module 29 is preferably the spinal and upper cervical impulse device disclosed in U.S. Pat. No. 8,152,747, which is incorporated herein in its entirety. As shown in FIGS. 4 and 5, the details of steps 42 and 44 above are as follows:

Three x rays are used: 1) frontal; 2) top skull; and 3) lateral image. As shown in FIG. 4, the attending professional selects frontal view 80 and then clicks 100 on the right inferior tip caudal edge of the mastoid process and the left inferior tip caudal edge of the mastoid process. The client software or the remote server processes the data and an adjustable grid appears 102 connecting the mastoid points. The width and height of the grid can be adjusted 105 using the slide bars and the tilt/angle of the grid can be adjusted 105 by dragging the end point of the line on the base of the grid. The attending professional then clicks 104 on the left edge of the skull where the flatness starts and on the left edge of the skull where the flatness ends. The client software or the remote server processes the data and horizontal lines appear 106 across the skull, parallel to the mastoid line. The attending professional then clicks 108 on the right inferior margin of posterior arch (atlas) at the point of contact with the lateral mass and the left inferior margin of posterior arch (atlas) at the point of contact with the lateral mass. The client software or the remote server processes the data and an adjustable grid appears 110 connecting the 2 atlas points, defining the atlas tilt. The width and height of the grid can be adjusted 111 using the slide bars and the tilt/angle of the grid can be adjusted 105 by dragging the end point of the line on the base of the grid. The attending professional then clicks 112 on the right superior articular facet lateral margin, the right superior articular facet medial margin, the left superior articular facet lateral margin and the left superior articular facet medial margin. The client software or the remote server processes the data and an adjustable circle appears 114, fit to the 4 points. The circle can be adjusting by dragging any of the 4 points until it represents the atlas circle. The attending professional then clicks 116 on the right superior articular facet lateral margin, the right superior articular facet medial margin (Point of intersection between Dens axis and Corpus vertebrae axis), the left superior articular facet medial margin (Point of intersection between Dens axis and Corpus vertebrae axis) and the left superior articular facet lateral margin. The client software or the remote server processes the data and these points appear 118. Finally, they click 120 on the right lateral margin of C7 transverse process and the left lateral margin of C7 transverse process. The client software or the remote server processes the data and these points appear 122. As would be known to one skilled in the art, the attending physician can identify the points of interest in any order that allows for the appropriate data to be collected. The points, lines and circle that appear are stored as data 124.

Once the data have been entered, only the remote server can be used to determine 160 a treatment protocol based on the data (this is done once all the steps before step 160 in both FIGS. 4 and 5 have been completed). The data and treatment protocol are saved 162 on the remote server, along with the patient records, that include patient identifier, a health professional identifier, a location identifier, and authentication information. If a clinic is wanting to provide a treatment, they can make a request for the protocol to be sent to the treatment device at their clinic, by entering the relevant identifiers and the authentication information. In this manner, a standard, personalized treatment can be provided to the patient.

As shown in FIG. 5, the attending professional selects 126 points on the x ray as follows. Two symmetrical structures on the skull, and a left symmetrical point of both lateral margins of nasal structure are selected. The client software or the remote server processes 128 the data and a dotted line appears 130 to the right of the point, across the nasal structure. The attending physician clicks 132 on the point where the dotted line crosses the lateral nasal structure margin. The client software or the remote server processes the data and a right symmetrical point of both lateral margins of nasal structure is determined 134. Both the left and right lateral margin of the odontoid (Dens) are then selected 136 as are the lateral inferior margin of the left transverse process of C1 and the lateral inferior margin of the right transverse process of C1 The client software or the remote server processes the data and the points appear 138. The points and lines that appear are stored as data 124.

The attending professional then moves 140 to the lateral view x ray. On the Lateral Mode, they select 142 2 points: the anterior margin of the pituitary fossa; and the anterior-Inferior margin of the vertebral body of C5. Once the two points have been selected, they click 144 “Lateral” and a pop-up screen appears 146. They select “Yes” 148 and are returned 150 to the patient analysis screen. The professional enters 152 their name and any notes, then selects “Save”. The points and lines that appear are stored as data 124.

Once the data have been entered, only the remote server can be used to determine a treatment protocol 160 based on the data (this is done once all the steps before step 160 in both FIGS. 4 and 5 have been completed). The data and treatment protocol are saved 162 on the remote server, along with the patient records, that include patient identifier, a health professional identifier, a location identifier, and authentication information. If a clinic is wanting to provide a treatment, they can make a request for the protocol to be sent to the treatment device at their clinic, by entering the relevant identifiers and the authentication information. In this manner, a standard, personalized treatment can be provided to the patient.

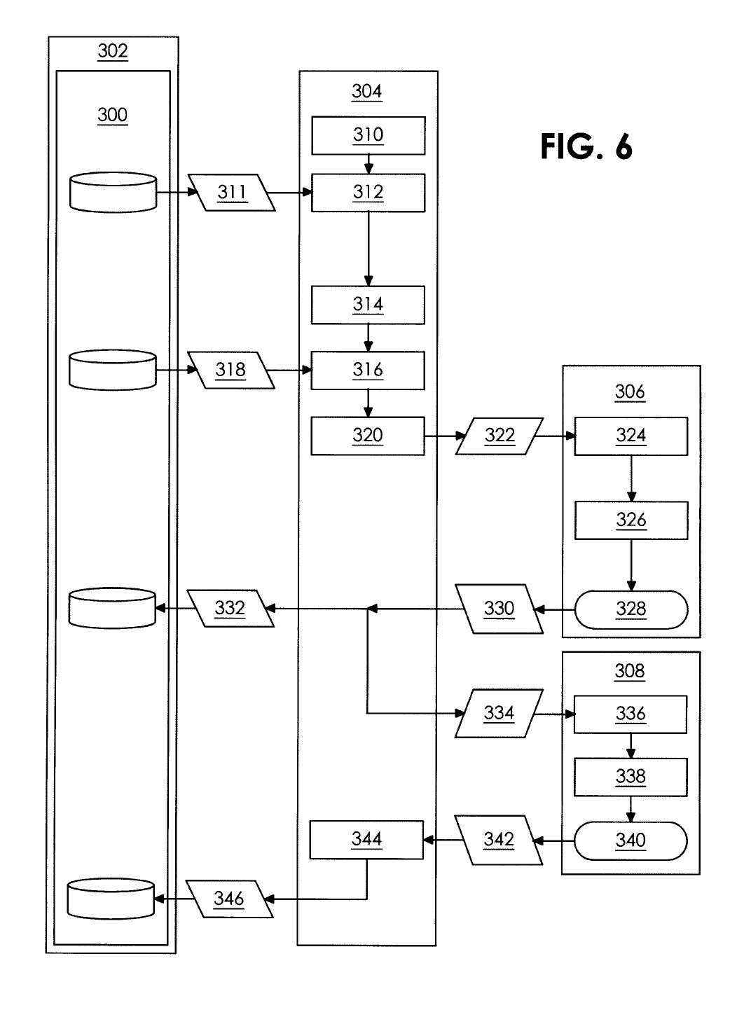

An overview of the architecture of the system is shown in FIG. 6. There is a database 300 in a database server 302, an application 304, a client 306 and an analytics server 308. A user launches 310 the application 304 on their computer. The login credentials 311 are sent from the database 300 and verified 312. If they are valid, the application 304 opens 314. The user navigates 316 the application 304 to select a patient. Patient data and x-rays are sent 318 from the database 300 to the application 304. The user then launches 320 the client 306 for the selected patient. Patient data and x-rays are sent 322 from the application 304 to the client 306. The client 306 displays 324 the patient x-rays. The user performs 326 the analysis of the x-rays by mapping the point coordinates. The client closes 328. The point coordinates are sent 330 to the application 304 and then are sent 332 to the database 300. The point coordinates are then sent 334 to the analytics server 308. The server 308 receives 336 the point coordinates and then calculates 338 the treatment parameters. The server closes 340 and sends 342 the treatment details to the application 304. The application 304 receives 344 the treatment parameters and sends 346 them to the database 300 for storage. The treatment protocol can then be retrieved and used on that patient at any location that has access to the database 300. The database 300 is segregated from the analytics server 308.

The foregoing technology can be best understood by way of the following examples.

### Example 1

By way of example, a suitable exemplary process is as follows. A new patient is assessed at a treatment centre A in Vancouver BC. The x-rays are taken, the professional identifies, by clicking, points on the x rays. The client software creates data on the basis of the inputs, and the data are entered into the application and analysed by the remote server software. The patient data are stored in the server, which is located at a remote server location. The analysis results in a treatment protocol which is also stored on the remote server. The data and treatment protocol are associated with treatment centre A. The treatment protocol is then used in treating the patient, a use is recorded and the service fee is recorded for treatment centre A. The patient returns for nine more treatments at treatment centre A and each time, the client communicates with the server, the patient data and treatment protocol are sent to the client, a treatment occurs and a service fee is recorded. The treatment protocol is sent to the treatment device. The treatment centre is then billed for the treatments.

### Example 2

By way of example, a suitable exemplary process is as follows. A new patient is assessed at a treatment centre A in Vancouver BC. The x-rays are taken, the professional identifies, by clicking, points on the x rays. The client software creates data on the basis of the inputs, and the data are entered into the application and analysed by the remote server software. The analysis results in a treatment protocol which is also stored on the remote server. The patient data and treatment protocol are stored in the server, which is located at a remote server location. The data are associated with treatment centre A. The treatment protocol is then used in treating the patient, a use is recorded and the service fee is recorded for treatment centre A. The patient returns to treatment centre A for two more treatments and then moves to Toronto. The patient goes to treatment centre B for seven more treatments. When they arrive at the centre, the professional logs onto the client and retrieves all the information on the patient from the server. The treatment protocol is sent to the treatment device. The patient receives the same treatments that they would have received had they stayed in Vancouver and attended treatment centre A. The royalties are recorded for each treatment at treatment centre B. Each centre is then billed for the treatments done at their centre. Treatment is seamless between the centres.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize, or be able to ascertain using no more than routine experimentation, many equivalents to the specific example embodiments specifically described herein. For example, other imaging techniques may be used, resulting in other images being analysed. Such equivalents are intended to be encompassed in the scope of the claims, if appended hereto or subsequently filed.

TABLE 1
Examples of the measurements generated by the application software:

| | | | | | | Atlas | Atlas | | Atlas |
|---|---|---|---|---|---|---|---|---|---|
| | Skull | Skull | Atlas | Atlas | Atlas | top | bottom | Linear | rotation |
| Patient | tilt | degree | tilt | contact | direction | radius | radius | vector | magnitude |
| A | right | 1.65 | right | 3.1 | right | 5.625 | 8.75 | 1.06 | 0.42 |
| B | right | 1.17 | left | 1.7 | left | 6.196 | 6.196 | −.5 | 4.28 |
| C | left | 2.62 | left | 1.4 | left | 4.851 | 5.063 | −.394 | 3.2 |
| D | left | 1.31 | right | .57 | right | 5.41 | 7.0 | .297 | 2.63 |
| E | right | 1.14 | left | 1.5 | left | 5.47 | 6.86 | .194 | .073 |

1. A method for monetizing a treatment protocol, the method comprising: a remote server receiving a request for a patient record from a health care provider, the remote server comprising a database; allowing the health care provider access to the patient record; receiving a request for a treatment protocol from the health care provider; the remote server providing the treatment protocol to a treatment device; the treatment device conducting the treatment protocol; and the remote server assessing the health care provider a fee for the treatment and recording the fee; wherein the database is partitioned such that the patient record and the treatment protocol are retained in separate parts of the database and each part has different access control criteria thereby restricting the health care provider's access to the treatment protocol.

2. The method of claim 1, wherein receiving the request comprises receiving a patient identifier, a health care provider identifier, a location identifier, and authentication information; allowing access comprises processing the identifiers and authentication information and providing access based on the access control criteria; and providing the treatment protocol comprises identifying the treatment device based on the location identifier and selectively communicating the treatment protocol to the treatment device.

3. The method of claim 1, wherein there is more than one health care provider in more than one location, and the database is partitioned such that the health care providers share access to the patient data, cannot access the treatment protocol, and can only access their own assessed fees, based on different access control criteria.

4. The method of claim 3, wherein the fee is a service fee.

5. The method of claim 4, further comprising the health care professional inputting patient data into a client and the client sending the data to an analytics server for processing into the treatment protocol prior to a request being made, wherein inputting patient data comprises identifying on a user interface, a plurality of points on frontal, side and top x ray images of the patient's skull.

6. The method of claim 5, further comprising determining a mean axis of rotation.

7. The method of claim 5, wherein the treatment device is a spinal and upper cervical impulse device.

8. A system configured to monetize a treatment protocol requested by a health care professional, the system comprising: a communication link; a treatment device; a remote server for receiving a request from the health care professional for a patient record, providing the patient record to the health care professional and providing the treatment protocol to the treatment device via the communication link, the remote server comprising a database and a processor, wherein the database is partitioned such that the patient record and the treatment protocol are retained in separate parts of the database and each part has different access control criteria thereby restricting the health care professional's access to the treatment protocol; a client, the client comprising a user interface and a communication module for communication with the remote server via the communication link, to allow the health care professional to send the request to access the patient record, to send a request to the remote server for the treatment protocol and to receive a bill associated with the client for the treatment.

9. The system of claim 8, wherein the client is configured to provide a patient identifier, a health care professional identifier, a location identifier, and authentication information and the processor in the remote server is configured to process the identifiers and authentication information, provide access based on the access control criteria, identify the treatment device based on the location identifier and selectively communicate the treatment protocol to the treatment device.

10. The system of claim 9, wherein the database is partitioned such that more than one health care professional shares access to the patient data, cannot access the treatment protocol, and can only access their own fees assessed, based on different access control criteria.

11. The system of claim 10, wherein the processor is configured to assess a service fee.

12. The system of claim 11, further comprising an analytics server, for processing the patient data into the treatment protocol.

13. The system of claim 12, wherein the user interface comprises a display and a mouse for identifying a plurality of points on frontal, side and top x ray images of a patient's skull.

14. (canceled)

15. (canceled)

16. The system of claim 13, wherein the communication link is a wireless communication link.

17. The system of claim 16, wherein the wireless communication link is the internet.

18. The system of claim 17, wherein data are stored in a cloud.

19. A method of monetizing a treatment protocol, the method comprising: identifying a patient with patient identifiers; collecting data from x-rays of a patient by a user clicking on a series of landmarks in the x-rays; sending the patient identifiers and data to an analytics server for processing; processing the data to determine a confidential treatment protocol; associating the confidential treatment protocol with the patient identifiers and the user; storing the confidential treatment protocol in a remote database; providing the confidential treatment protocol to a treatment device in response to the user's request; recording the treatment at the remote database and assessing a fee to the user in response to the treatment, wherein the database is partitioned to allow the user access to only the patient identifiers, patient data and a record of the number of treatments.

20. The method of claim 19 wherein the series of landmarks are a plurality of points on frontal, side and top x ray images of the patient's skull.

21. The method of claim 20, further comprising determining a mean axis of rotation.

22. The method of claim 21, wherein the treatment device is a spinal and upper cervical impulse device.

23. (canceled)

24. (canceled)

25. (canceled)

26. (canceled)
